(19) United States
(12) Patent Application Publication
(10) Pub. No.: US 2007/0197992 A1
(43) Pub. Date: Aug. 23, 2007

(54) Absorbent article with urine-permeable coversheet

(75) Inventors: Cornelia Beate Martynus, Nidderau (DE); Maja Wciorka, Schwalbach (DE); Ralf Geilich, Eppstein (DE); Joseph Allen Eckstein, Sunman, IN (US); David James Dahlinger, Mason, OH (US); Thomas Friedrich Frisch, Schwalbach (DE)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/701,152

(22) Filed: Feb. 1, 2007

Related U.S. Application Data
(60) Provisional application No. 60/764,228, filed on Feb. 1, 2006.

(51) Int. Cl. A61F 13/15

(52) U.S. Cl. 604/385.19; 604/385.09

(57) ABSTRACT

The present disclosure is directed to an absorbent article (e.g., a diaper or training pant) having a backsheet, an absorbent core and a topsheet provided with at least one opening adapted to receive fecal material. The article also includes a genital coversheet with one or more central longitudinal (partial) folds or fixed folds, which in use forms a pocket to cover the genitals, and which is positioned in, under or above part of the opening, such that a void space can be created between the genital coversheet and the absorbent core and such that a void space is present between the topsheet and the absorbent core. The present disclosure also relates to process for making pocket-shape genital coversheets for absorbent articles of the invention.

### CROSS REFERENCE TO RELATED APPLICATION

This application claims the benefit of U.S. Provisional Application No. 60/764,228, filed on Feb. 1, 2006, which is hereby incorporated by reference herein.

### FIELD OF THE INVENTION

The present disclosure relates to an absorbent article, such as a diaper or training pants, having a backsheet, an absorbent core and a topsheet provided with at least one opening adapted to receive fecal material. The absorbent article includes a genital coversheet with one or more longitudinal (central) folds or partial folds, which in use forms a pocket to cover the genitals, and which is positioned in, under or above part of the opening, such that a void space is present between the topsheet and the absorbent core. The present disclosure also relates to processes for making pocket shaped genital coversheets for use in absorbent articles, as described herein.

### BACKGROUND OF THE INVENTION

It is well known that fecal material is often difficult to remove from the skin of the user, in particular on sensitive skin such as by young babies and the skin around the genitals. Moreover, it is well known that fecal material on the skin can cause irritation and redness of the skin and some times even dermatitis of the skin.

One of the solutions to reduce the fecal material on the skin is to provide a means to isolate the fecal material immediately after discharge, away from the skin. For example, diapers with a topsheet with one or more openings, through which the feces can pass to a void space between the topsheet and the absorbent core, have been developed. The fecal material is then stored underneath this topsheet, away from the skin.

However, the inventors have found that occasionally still some fecal material may transfer back onto the skin including on the genitals.

Thereto, the inventors have developed an absorbent article with a genital coversheet, which protects in use the genitals from being soiled by the fecal material. In some embodiments, this cover may only be present in the areas of the topsheet and of the opening, which are in proximity with the genitals (i.e., the front of the opening in the topsheet of the diaper), to allow sufficient open space to pass the fecal material through. The cover may be an integral part of the topsheet with the opening, or it may be an additional component (sheet) attached to, for example, the topsheet with the opening.

The genital coversheet may include one or more folds such forming a pocket in use, which can partially enclose and/or cover the genitals.

### SUMMARY OF THE INVENTION

Aspects of the present disclosure provide an absorbent article having a backsheet, an absorbent core and a topsheet provided with at least one opening adapted to receive fecal material, the topsheet and the opening thereof each having a front region and a back region. The absorbent article may also include a genital coversheet having one or more folds or partial folds, for example, a single partial central fold, which form a pocket that can enclose and/or cover the genitals in use, and which is positioned in, under or above the front region of the opening. A void space is formable between the genital coversheet and the absorbent core, and a void space is present between the topsheet and the absorbent core. Thus, the genital coversheet reduces the size of the opening.

The folds may be longitudinal folds or partial folds, and the folds may be fixed folds or partial folds, as described herein.

The present disclosure also relates to processes for making a pocket-shape genital-coversheet suitable as a high speed process.

The genital coversheet may be urine permeable, so urine can pass through it to the absorbent core of the diaper. The genital coversheet may have a low rewet, so the amount of urine passing back to the genitals is minimized.

The absorbent article may have a topsheet and/or the genital coversheet that is colored or opaque to mask any fecal material deposited under the topsheet and/or under the genital coversheet.

### BRIEF DESCRIPTION OF THE DRAWINGS

FIG. 1 shows a perspective view of an absorbent article including a genital coversheet.

FIG. 2 shows a plane top-view of the absorbent article of FIG. 1.

FIG. 3 shows a perspective cross-sectional view of an absorbent article taken along line III-III in FIG. 2.

FIGS. 4 and 5 show a topsheet with a slit opening therein.

FIG. 6 shows a perspective view of a genital coversheet before implementation in the absorbent article.

FIG. 7A shows a perspective view of a genital coversheet, folded for implementation in the absorbent article.

FIG. 7B shows a perspective view of another genital coversheet, folded for implementation in the absorbent article.

FIG. 7C shows a perspective view of another genital cover sheet, folded for implementation in the absorbent article.

FIG. 7D shows a perspective view of another genital cover sheet, folded for implementation in the absorbent article.

### DETAILED DESCRIPTION OF THE INVENTION

As used herein, the following terms have the following meanings:

As used herein, “absorbent article” means any article that can absorb body fluids and is suitable to be placed in close proximity to the genitals of the user, including in particular adult or infant diapers and so-called training or pull-up pants.

As used herein “front region” and “back region” refer to the two regions, which are in use, respectively, closest to the front of the wearer and the back of the wearer.

As used herein “crotch side portion” is the portion of the topsheet between the longitudinal side edge of the opening and the longitudinal side edge of the topsheet, and the crotch side portion may thus be part of the front region and/or the back region.

As used herein, the term “void space” is a cavity in the article present in at least the relaxed state, which serves to accept and contain bodily exudates such as fecal material, for example, at least 3 or even 5 cm³ in relaxed state.

When used herein, “longitudinal” is the direction running substantially parallel to the maximum linear dimension of the component, typically to the longitudinal axis of the article, and includes directions within 30° of this parallel, when applicable.

The “lateral” or “transverse” direction is orthogonal to the longitudinal direction and in the same plan of the majority of the article and the longitudinal axis and includes directions within 30° of the orthogonal, when applicable.

As used herein, the term “attached” encompasses configurations wherein a first element is directly secured to another element by affixing the element directly to a second element.

As used herein, the term “joined” or “connected” encompasses configurations wherein a first element is indirectly secured to a second element by affixing the first element to a third, intermediate member(s), which in turn are affixed to the second element.

As used herein “stretched” or “stretched state”. means that the article or topsheet thereof is stretched to its maximum length, which is typically determined by the length of a non-elastically extendable component which is part thereof, e.g., the backsheet or the non-elastically extendable material of the topsheet.

As used herein “relaxed” or “relaxed state” means the state where no forces are applied to the article or component thereof (other than naturally occurring forces such as gravity), when the article is laid on a horizontal surface, such that the transverse front and back edge are flat on the horizontal surface and the transverse center line or axis is on the horizontal surface.

As used herein, “elasticated” means typically, that the component consists of or comprises elastic material, which is elastic in at least one direction. “Non-elasticated” when used herein means that the component does not comprise any elastic material.

As used herein, “along” means “at least partially substantially parallel to and adjacent to”. Adjacent includes “in close proximity with” and “in contact with”.

As used herein, “opening in the topsheet” means an area completely circumscribed by the topsheet, but where the topsheet material is not present, and which is large enough to receive fecal material, for example, being at least 2 cm long or wide, or having a surface area of at least 2 cm².

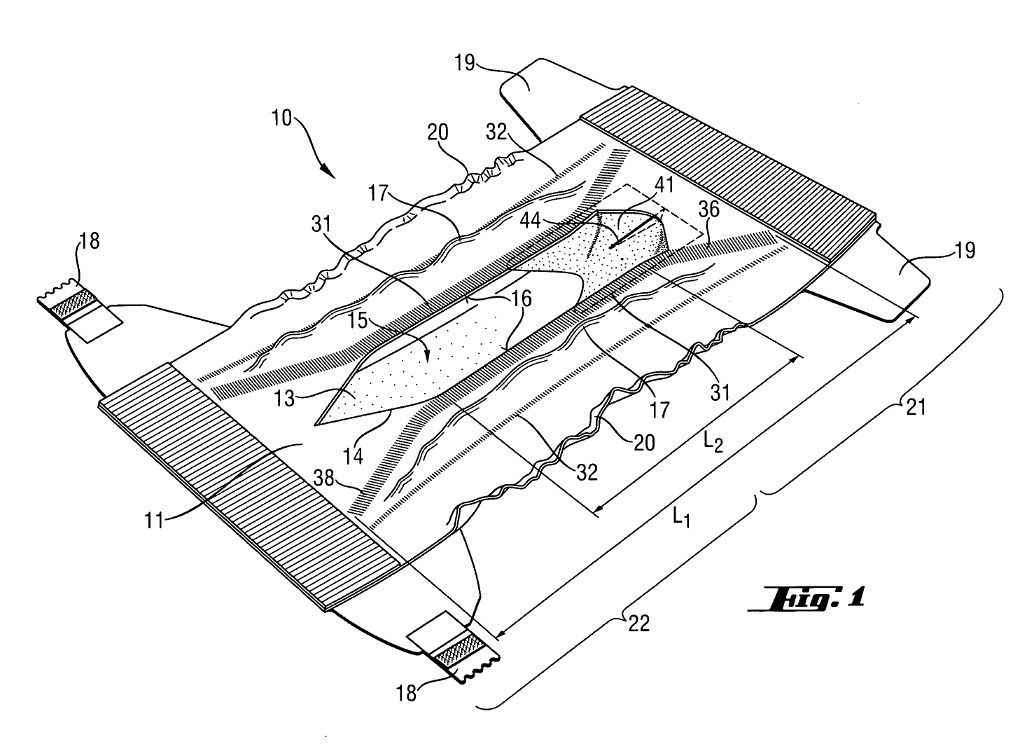

Absorbent articles according the present disclosure may include one or more openings for the reception of fecal material and a genital coversheet. The opening may be in the form of a slit opening, and in some embodiments, a single opening may be used. FIGS. 1-3 show an absorbent article 10 in the form of a diaper including a backsheet 12, an absorbent core 13, and a topsheet 11. The longitudinal side edges of the topsheet 11 may be joined or attached to the longitudinal side edges of the backsheet 12, by any attachment means known in the art, to form longitudinal opposing attachment areas. In certain embodiments, the topsheet 11 and the backsheet 12 are attached directly to one another in some locations and are indirectly joined together in other locations. As discussed in more detail below, the topsheet 11 is provided with at least one opening 14 adapted to receive fecal material. The topsheet 11 and the opening each have a front region 21 and a back region 22. The opening 14 may be present in (part of) the front region of the topsheet (in use towards the front of the user) and in (part of) the back region of the topsheet. The absorbent article also includes a genital coversheet 41 positioned under the topsheet 11 and under the opening 14 in the topsheet. It is to be appreciated that the genital coversheet may be positioned in, under, or above the front region of the opening 14. As discussed in more detail below, the genital coversheet 41 may include one or more folds or partial folds to form a pocket that in use cover the genitals. As shown in FIGS. 1-3, a void space 15 is present between the absorbent core 13 and the topsheet 11 and between the absorbent core 13 and the genital coversheet 41.

The diapers herein may have a fastening system, which may be joined to the waistband, as known in the art. Some fastening systems include fastening tabs and landing zones, wherein the fastening tabs are attached or joined to the back region of the diaper and the landing zones are part of the front region of the diaper. For example, as shown in FIGS. 1-3, the diaper 10 includes a back waist band with ears with fasteners 18 and a front waist band 19 with receiving areas for the fasteners. In some configurations, the fasteners comprise hooks and/ or adhesive and the receiving areas may be formed from loop-containing material.

The absorbent article herein may be a disposable adult or infant diaper or training pants/pull-up pants. Diapers or training pants may have side panels, and/or one or more pairs of elasticated leg cuffs that provide improved containment of liquids and other body exudates. Leg cuffs may also be referred to as leg bands, side flaps, barrier cuffs, or elastic cuffs, as described in; U.S. Pat. No. 3,860,003; U.S Pat. No. 4,808,178 and U.S. Pat. No. 4,909; U.S. Pat. No. 4,695,278 and U.S. Pat. No. 4,795,454. As shown in FIGS. 1-3, the diaper 10 includes elasticated bands along the longitudinal side edges of the diaper 10, so called leg cuffs 20.

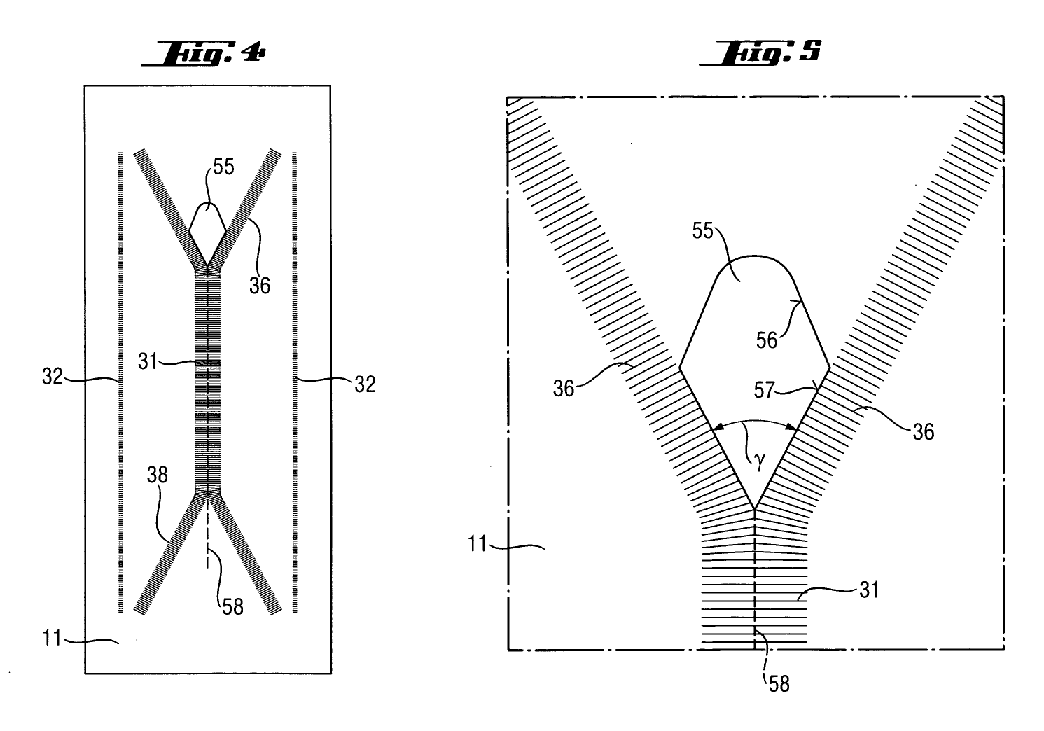

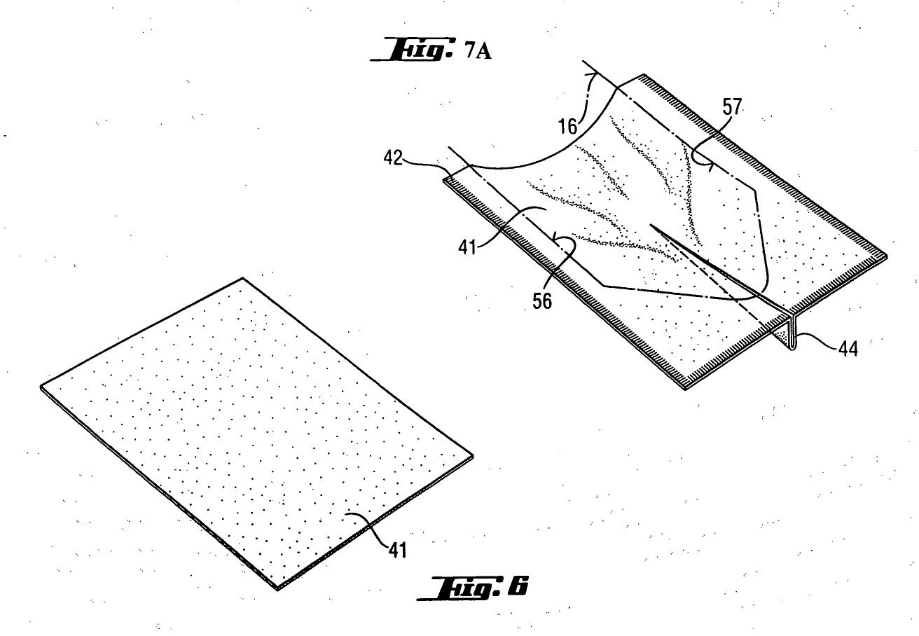

As previously mentioned, the topsheet 11 may be provided with at least one opening 14 adapted to receive fecal material. The topsheet 11 shown in FIGS. 4 and 5 may be made by making a longitudinal slit 58 (optionally with no width dimension) in the topsheet material and cutting an additional front cut out section 55 out of the topsheet, the section 55 extending from the front of the slit 58 towards the front of the diaper 10. The front cut out section 55 may be “diamond” shaped, although the “diamond” shaped cut out section 55 has a front edge 56, which is curved. The side edges 57, extending directly from the slit 58 to the front edge of the diaper 10 and connecting the curved front edge 56 with the slit 58, may be connected to one another with an angle γ, which may be between 40° and 140° or between 55° and 110°.

The exact shape of the opening(s) 14 may vary, depending on the size of the topsheet 11 and/or the absorbent article 10. For example, in one embodiment the opening is in the form of a slit opening with substantially parallel longitudinal side edges, which are connected in the front and back by V-shaped or rounded V-shaped, as shown in FIGS. 1-4, front and back edges, wherein both the front and back V-shaped edges comprise two angled edges. In some embodiments, the back V-shaped edges may have a larger angle than the front V-shaped edges. The front V-shaped edges may have an angle of 20° to 100° or, alternately, from 45° to 65°, as shown in FIGS. 1 and 4. The slit opening may optionally extend into an additional cut-out area which is, for example, diamond shaped, as described herein.

The dimensions the opening(s) 14 may also vary, depending on the size of the topsheet 11 and/or the absorbent article 10. In some embodiments, the topsheet may have a slit opening having a longitudinal dimension (length) substantially parallel to the longitudinal axis of the topsheet 11 and of the diaper 10. In stretched state, the opening (or openings) of the topsheet may be configured such that from 20% to 40% or from 20% to 30% of the length of the opening (or total length of the openings) extends from the transverse axis of the topsheet towards the front edge of the topsheet, and the remaining percentage extends towards the back edge of the topsheet. In some embodiments, the maximum length of the slit opening may be about 40% to 90%, about 50% to 80%, or about 60% to 70% of the total length L of the absorbent article. In one example, a size 4 diaper may have a maximum topsheet length of between 45 cm and 55 cm or between 48 cm and 52 cm. In some embodiments, the length of the single slit opening, when the diaper is in stretched state, may be from 20 cm to 40 cm; from 25 cm to 35 cm; or from 28 cm to 32 cm. In some embodiments, the average width of the opening, in stretched state, may be from 5% to 30% or 10% to 25% of the average width of the topsheet (including opening width). In one example, a size 4 diaper may have an average width of the opening of from 15 mm to 60 mm or from 20 mm to 40 mm.

As shown in FIG. 1, the topsheet 11 may include a primary elasticated area 31 adjacent to or in close proximity with each longitudinal side edge 16 of the opening 14 to form a pair of opposing, elasticated areas. In some embodiments, the primary elasticated areas may extend from the side edges 16 of the opening(s) 14 towards or completely to the front and back edge of the topsheet 11. Thus, the primary elasticated areas may be longer than the opening 14. The elasticated area may be positioned over the full length of the topsheet, or at least the part of the topsheet which in use is intended to receive body exudates (e.g., the topsheet minus the parts thereof which form (part of) the waist bands). An elasticated area in the topsheet may be formed from a multitude of thin strands of elastic material or, for example, from a single band of elastic material. As discussed below, the absorbent article may also include secondary elasticated areas in each crotch side portion (i.e., the portion of the topsheet between the longitudinal side edge of the topsheet 11 and the longitudinal side edge of the opening 14). Each secondary elasticized area may have an overall curvature, curving away from the primary elasticated area of the same crotch side portion.

As shown in FIG. 1, the primary elasticated areas 31 may be positioned along the longitudinal side edges 16 of the opening 14. The topsheet 11 may also have secondary elasticated areas 32, or even tertiary elasticated areas (not shown). The primary elasticated areas 31 have each a central region with a length L2, the central regions being substantially parallel to one another, whereby L2 may be about 30% to 70% of the total length L1 of the primary elasticated areas 31. In some embodiments, L2 is about 40% to 80% of the maximum length of the opening 14. The primary elasticated areas 31 may have an X-shape, whereby the front end portions 36 bend away from one another and the back end portions 38 bend away from one another. The primary elasticated areas may also be parallel, such as described in copending application EP-A-1201212.

The primary elasticated area may be shaped such that it has a central portion that is substantially parallel to the central portion of the opposing primary elasticated area. As discussed above, the central portion has a length L2, which in some embodiments, may be 30% to 70% of the total length L1 of a corresponding elasticated area, and may be about 40% to 80% of the maximum length of the opening. In some embodiments, the total length of the elasticated area may be about 70% to 90%, about 80% to 90%, or about 85% of maximum length of the topsheet. The length of the primary elasticated area may also depend on the size of the topsheet 11 and/or the article 10. For example, for a size 4 diaper as described above the average length of the elasticated area in stretched state, may be at least 35 cm or from 35 cm to 45 cm. The width of the elasticated areas on the topsheet may also vary, depending on the exact dimensions of the topsheet 11 and/or the article 10. For example, for size 4 diapers as described above, a primary elasticated area, in stretched state, may be an elastic band, or a multitude of elastic strands, that has an average width of about 3 mm to 50 mm, about 3 mm to 40 mm, about 3 mm to 20 mm, or about 5 mm to 20 mm.

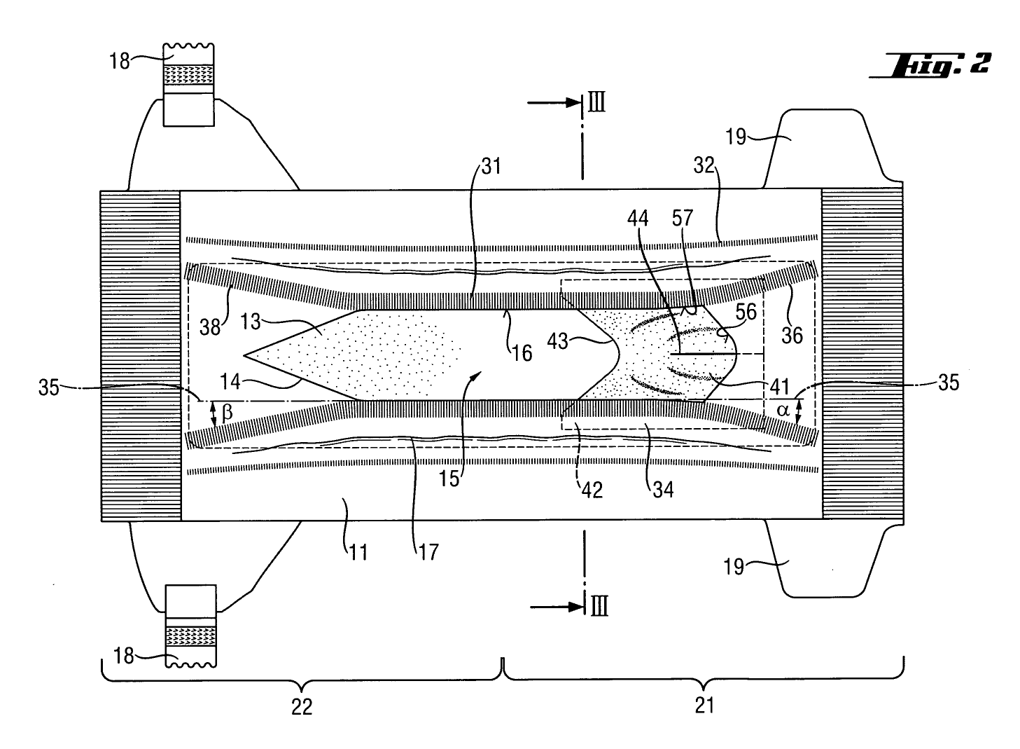

The front end portions of two opposing primary elasticated areas may bend away from one another (in the plane of the topsheet), so that the distance between the end edges of the opposing front end portions of two opposing elastic areas is larger that the distance between the central portions of two opposing elastic areas, and equally, the distance between the end edges of the opposing back end portions of two opposing elastic areas is larger that the distance between the central portions of two opposing elastic area. For example, as mentioned above and as shown in FIG. 2, the primary elasticated areas 31 may be in the shape of an X, whereby each front end portion 36 of the elasticated area has an angle α with the longitudinal line 35 parallel to the longitudinal axis of the topsheet 11 and through that part of the elasticated area that is (directly) adjacent a longitudinal side edge 16 of the opening. In some embodiments, this angle α may be about 17° to 30° in stretched state. In some embodiments, each back end portion 38 of the elasticated area may have an angle β which may be about 17° to 30° in stretched state.

In some embodiments, the front end portion of a primary elasticated area may have an angle with a longitudinal line through the central portion of the elasticated area and parallel to the longitudinal axis of the topsheet, the angle may be between 10° and 40°, between 17° to 35°, or between 20° and 35°. In other embodiments, the back end portion of each of the primary elasticated area may have an angle with a longitudinal line through the central portion of the elasticated area and parallel to the longitudinal axis of the topsheet. In some embodiments, the angle may be between 10° and 40°, between 17° to 35°, or between 20° and 35°. When both front end portions and both back end portions have an angle as above, the primary elasticated areas have, as is herein referred to, an X-shape, and a suitable X-shape is exemplified in FIGS. 1 and 2.

In some embodiments, the front end and/or the back end and/or the central portion of an elasticated area may be curved rather than straight. In such an embodiment, the angles above may be determined by the angle of the tangent line through the center point of the front end and/or back end, with the line parallel to the longitudinal axis of the topsheet and tangent to the center point of the central portion of the elasticated area.

The elasticated areas herein may be formed by attaching an elasticated material in stretched state or in a partial stretched state to the topsheet or to one or more carrier materials that are then subsequently attached to the topsheet. The elastic materials may be in the form of a multitude of strands or a single band with an average thickness (e.g., gauge) of at least 20 microns, at least 40 microns, or at least 60 microns. In some embodiments, the elastic material has an average thickness up to about 300 microns, up to 200 microns, or up to 150 microns. Suitable materials may have an average thickness of about 70 to 100 microns. Suitable elastic materials used herein may include VFE-CD, available from Tredegar, and L-86, L-89, or L-90, available from Fulflex (Limerick, Ireland).

The absorbent article 10 may be sag-tolerable and may include a topsheet 11 that is sag-tolerable. This means that the topsheet does not sag when the backsheet and absorbent core sag due to increased weight of the body exudates received by the article. In addition, the topsheet keeps its z-direction alignment with the anal region and genitals of the wearer, and may also keep its x and y direction alignment. The absorbent article (e.g., diaper or training pants) may include a means to ensure that the topsheet stays in about the same contact or close proximity with the wearer's anal and/or genital region when the backsheet and core sag, compared to just after application of the article to the wearer, when the backsheet and core do not yet sag. In some embodiments, the topsheet is sag-tolerable such that when the geometrical center point of the backsheet is pulled down 4 cm, (i) the topsheet does not move down more than 0.5 cm, more than 0.25 cm, or does not move down at all, and/or (ii) the longitudinal side edges of the opening do not move in the x and y direction more than 0.5 cm, more than 0.25 cm, or do not move at all.

In some embodiments, the topsheet 11 may be sag-tolerable and thereto non-elastically extendable and may have thereto one or more transverse folds and/or longitudinal folds 17, as shown in FIGS. 1-3. In some configurations, the average width of the topsheet 11, including the width of the opening 14, may be larger than the average distance between the longitudinal attachment areas of the topsheet 11 to the backsheet 12. In other configurations, the average width of the topsheet, including the width of the opening, may be larger than the average width of the backsheet. As such, the topsheet may, for example, have one or more transverse and/or longitudinal folds, which can unfold in use and allow sagging of the core and backsheet, while the topsheet remains in place. In some embodiments, the topsheet 11 with the longitudinal folds 17 is not attached to the absorbent core 13, but directly to the backsheet 12 with longitudinal attachment lines 23, to ensure that the diaper 10 and the topsheet 11 thereof are sag-tolerable.

The topsheet herein may be liquid or urine pervious or impervious. The topsheet may be liquid or urine pervious in one direction, but liquid or urine impervious in the opposite direction (e.g., that body fluids may penetrate through the topsheet to the remaining part of the diaper, but that no or limited amounts of liquid (urine) can penetrate in reverse direction, towards the wearer's skin). In some embodiments, the topsheet or at least more than 50% of its surface area (that faces the wearer in use) may be hydrophobic. The topsheet may be urine impermeable and feces impermeable. The topsheet may be made of a material having a Strike Through time for the first gush of more than 120 seconds, as determined by the test method described below. Topsheets may be considered urine-impermeable and feces impermeable when they have a low surface energy and a uniform pore size distribution. Low surface energy values, pore sizes, and air permeability values are described in co-pending application EP-A-1417945. Some topsheets may comprise materials having an alcohol repellency of at least 7, at least 8, at least 9, or at least 10; having a surface energy of less than 25 mN/m; having a contact angle with water of above 130°; and/or having a mean pore size of less than 20 microns, less than 17 microns, or less than 13 microns, but generally at least 2 microns or at least 5 microns. Some topsheets may have an air permeability of at least 3 Darcy, at least 10 Darcy, at least 20 Darcy, at least 30 Darcy, or at least 50 Darcy.

Some topsheets may be made of hydrophobic material or may treated to be hydrophobic (in order to isolate the wearer's skin from liquids contained in remaining part of the diaper), with, for example, a hydrophobic surface coating such as described in co-pending application U.S. 60/543,785, filed Feb. 11, 2004. The hydrophobic surface coating may include one or more silicone polymers or fluorinated polymers. Silicone polymers may, for example, be selected from the group consisting of silicone MQ resins, polydimethysiloxanes, crosslinked silicones, silicone liquid elastomers, and combinations thereof. In some embodiments, the molecular weight of such silicone polymers may be at least about 4000 MW, at least about 10,000 MW, at least about 15,000 MW, at least about 20,000 MW, or at least about 25,000 MW. Polydimethylsiloxanes may be selected from the group consisting of vinyl-terminated polydimethsiloxanes, methyl hydrogen dimethylsiloxanes, hydroxyl-terminated polydimethysiloxanes, organo-modified polydimethylsiloxanes, and combinations thereof. Fluorinated polymers may be selected from the group consisting of telomers and polymers containing tetrafluoroethylene and/or perfluorinated alkyl chains. For instance, fluorinated surfactants, which are commercially available from Dupont under the tradename Zonyl®, may be suitable for use herein. In particular, Zonyl®321, 329, 8740, 9027, and 9360 may be suited for use. Additionally, other Zonyl® materials include fluroadditives like micro-powders may be useful herein. These include, but are not limited to Zonyl® MP1100, MP1200, MP1400, MP1500J, MP1600N, TE-3667N (which is a water dispersion). In certain embodiments, the coating is free of aminosilicones. These materials may be deposited onto the topsheet in amounts of from at least about 0.01 gsm (gram of material/square meter of topsheet), at least about 0.05 gsm, or at least about 0.1 gsm.

The topsheet may be manufactured from a wide range of materials, including woven or non-woven webs of natural fibers (e.g., wood or cotton fibers) or synthetic fibers (e.g., polyester, polyethylene and/or polypropylene fibers), or a combination of natural and synthetic fibers. If the topsheet includes fibers, the fibers may be, for example, spun bond, carded, wet-laid, melt blown, hydro entangled, or otherwise processed as is known in the art. Suitable materials may be compliant, soft feeling, and non-irritating to the wearer's skin. In some embodiments, suitable materials include webs comprising spunbond layers (S) and meltblown layer(s) (M), whereby the surfaces of the web are formed by spunbond layer(s). The webs may have a relatively high basis weight, for example, more than 25 gram/m² (gsm). Suitable webs may include, for example, 34 gsm SMMS (whereby 12 gsm meltblown and 5 gsm spunbond); 34 gsm SMMS (whereby 10 gsm meltblown and 7 gsm spunbond); 30 gsm SMMS (whereby 10 gsm meltblown and 5 gsm spunbond); 30 gsm SMMS (whereby 8 gsm meltblown and 7 gsm spunbond); 34 gsm SMS (whereby 20 gsm meltblown and 7 gsm spunbond), or, for example, webs comprising two layers of 17 gsm SMMS.

Any portion of the topsheet may be coated with a lotion or powder, known in the art. In some embodiments, the lotion is present on the primary elasticated areas and/or on the secondary elasticated areas. The lotion used on one elasticated area may be different to the lotion used on another elasticated area, or on the remaining part of the topsheet. Examples of lotions include those described in U.S. Pat. No. 5,607,760; U.S. Pat. No. 5,609,587; U.S. Pat. No. 5,635,191; U.S. Pat. No. 5,643,588; and WO 95/24173, provided the lotion is compatible with the elastic material, and does not destroy the elastic material or reduce its elasticity.

As previously mentioned with reference to FIGS. 1-3, the absorbent article 10 also includes a backsheet 12, as known in the art. The backsheet 12, or any portion thereof, may be elastically extendable in one or more directions. The backsheet 12 may be attached or joined to the topsheet 11, the absorbent core 13, or any other element of the diaper 10 by any attachment means known in the art. The longitudinal side edges of the topsheet and backsheet may be directly attached to one another, but that the longitudinal edges of the topsheet and the core are not attached to one another. The attachment means to attach the topsheet and the backsheet, but also the genital coversheet herein may include a uniform continuous layer of adhesive, a patterned layer of adhesive, or an array of separate lines, spirals, or spots of adhesive, such as disclosed in U.S. Pat. No. 4,573,986. Adhesives that have been found to be satisfactory are manufactured by H. B. Fuller Company of St. Paul, Minn. and marketed as HL-1620 and HL-1358-XZP. Alternatively, the attachment means may include heat bonds, pressure bonds, ultrasonic bonds, dynamic mechanical bonds, or any other suitable attachment means or combinations of these attachment means as are known in the art.

In some embodiments, the backsheet 12 may be liquid impervious and may comprises a thin plastic film, such as a thermoplastic film having a thickness of about 0.01 mm to about 0.05 mm. Suitable backsheet materials may include breathable material, which permit vapors to escape from the diaper while still preventing exudates from passing through the backsheet. Suitable backsheet films may include those manufactured by Tredegar Industries Inc. of Terre Haute, Ind. and sold under the trade names X15306, X10962 and X10964.

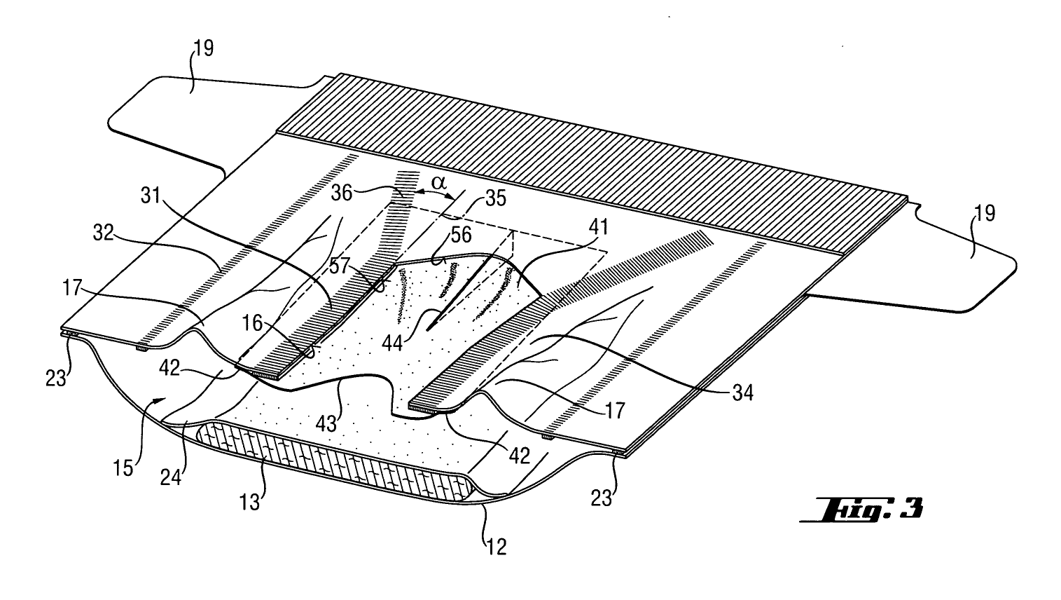

FIG. 3 shows a section view of the diaper 10 of FIGS. 1 and 2, according to line III-III in FIG. 2. It shows the void space 15 between the genital coversheet 41 and the absorbent core 13 and between the topsheet 11 and the absorbent core 13 and how the genital cover sheet 41 is formed into a pocket shape due to the fold 44 that is positioned into part of the void space 15 in use.

As mentioned above, the absorbent article may include an absorbent core. The absorbent core may comprise any absorbent material which is generally compressible, conformable, non-irritating to the wearer's skin, and capable of absorbing and retaining urine, such as comminuted wood pulp, creped cellulose wadding; melt blown polymers, including coform; chemically stiffened, modified or cross-linked cellulosic fibers; tissue, including tissue wraps and tissue laminates; absorbent foams; absorbent sponges; super absorbent polymers; absorbent gelling materials; or any other known absorbent material or combinations of materials; and may be absorbent cores which have an absorbent storage layer which comprises more than 80% by weight of the absorbent core content (e.g., excluding core wrap) of absorbent gelling material, and which may be free of airfelt.

The absorbent article may also include a sub-layer (which may be the same as the body facing liner described above) disposed between the topsheet and the absorbent core, capable of accepting, and/or immobilizing bodily exudates, typically fecal material. For example as shown in FIG. 3, the absorbent core 13 may comprise a specific sub-layer 24, which comprises means to immobilize fecal material, for example, a layer with vertically extending (z-direction) fibers, or an apertured web or film, as described herein. Suitable materials for use as the sub-layer may include large cell open foams, macro-porous compression resistant non woven highlofts, large size particulate forms of open and closed cell foams (macro and/or microporous), highloft non-wovens, polyolefin, polystyrene, polyurethane foams or particles, structures comprising a multiplicity of vertically oriented, which may be looped, strands of fibers, and/or apertured formed films, as described above with respect to the genital coversheet. (As used herein, the term “microporous” refers to materials that are capable of transporting fluids by capillary action, but having a mean pore size of more than 50 microns. The term “macroporous” refers to materials having pores too large to effect capillary transport of fluid, generally having pores greater than about 0.5 mm (mean) in diameter and more specifically, having pores greater than about 1.0 mm (mean) in diameter, but typically less than 10 mm or even less than 6 mm (mean).

As previously mentioned, the absorbent article includes a genital coversheet that may be present under, in, or above the opening in the topsheet. In use, the genital coversheet 41 will cover the genitals of the user. Typically, the genital coversheet 41 is such that it can form a pocket around the genitals. This is further shown in FIG. 3. The genital coversheet may be present under, in, or above that part of the opening that is in close proximity with the genitals during use (i.e., the front region of the opening). The genital coversheet may be attached to the side of the topsheet that faces away from the wearer, e.g., that faces the absorbent core.

The genital coversheet material may be attached to the absorbent article in such a manner that the longitudinal side edges of the genital coversheet 41 are substantially parallel and may also be parallel to the longitudinal axis of the diaper 10. Thus, the genital coversheet may have substantially parallel attachment areas that are parallel to the longitudinal axis of the article. Such an attachment area may be a single longitudinal attachment line or it may comprise of separate intermittent longitudinal attachment lines, or even separate step-wise applied intermittent attachment lines (that are each typically parallel to the longitudinal axis but positioned at a different distance from the longitudinal axis).

FIG. 2 shows a plane (top) view of the diaper 10 of FIG. 1, in stretched state. The genital coversheet 41 is placed under the topsheet 11 and attached along its longitudinal side edges to the topsheet 11, with attachment areas 42, which are, for example, in the form of longitudinal lines of adhesive. The attachment areas 42 may be positioned at least or only along either longitudinal side edges of the opening 16, and may also be parallel thereto and parallel to one another. Each attachment area 42 may be positioned between a primary elasticated area 31 and the longitudinal side edge of the topsheet, or between the primary elasticated area 31 and the secondary elasticated area 32 (when present), such that the genital coversheet 41 is attached to the topsheet 11 in at least or only the non-elasticated areas 34 of the topsheet 11.

When the genital coversheet is present under the topsheet and below the opening (i.e., between the topsheet and the absorbent core), the genital coversheet may not, in some embodiments, be attached to the absorbent core to ensure the creation of a maximum void space for fecal material between the topsheet and the absorbent core and between the genital coversheet and the absorbent core.

The genital coversheet may be attached to the absorbent article (e.g., the topsheet) by any means, including adhesive bonding, heat bonding, pressure bonding, and including various bonding patterns, such as a longitudinal line or a longitudinal line formed from individual transverse lines or dots, etc. The genital coversheet may be partially attached to the topsheet thereby forming attachment areas and non-attached area(s). One or more of the non-attached area(s) may form the protective genital cover. The genital coversheet may be attached to the topsheet by at least two attachment areas that are along, either directly adjacent or not, part of the longitudinal side edges of the opening. When the genital coversheet is attached to the topsheet with at least a pair of opposing attachment areas, the attachment areas in some embodiments do not overlap the primary elasticated areas, but, rather, are in close proximity to the primary elasticated areas.

The genital coversheet may be attached (e.g., to the topsheet) as described herein, with a pair of opposing attachment areas, with herein between one or more unattached area(s), as described above, whereby the average absolute width of the unattached area(s) of the genital coversheet, between the pair of attachment area(s), is larger than the average distance (width) between the two attachment areas, for example, by at least 1.2 times larger, or at least 1.5 times larger.

The topsheet may comprise attachment areas (where the genital coversheet is attached to the topsheet) along the opposing longitudinal side edges of the (slit) opening in the topsheet. The attachment areas may be spaced apart from these longitudinal side edges. The attachment areas may be spaced apart from these longitudinal side edges by about 2 to 20 mm or by about 5 to 15 mm.

In some embodiments, the maximum length of the part of the genital coversheet 41 that is present above, in or under the opening 14 is 10% to 50% of the maximum length of the opening. In other embodiments, the maximum length of the part of the genital coversheet that is present above, in or under the opening may be 10% to 30%, 13% to 28%, or 17% to 27% of the maximum length of the opening. In other words, in some embodiments, at the most 50% of the maximum length of the opening is ‘covered’ by the longest part of the genital coversheet, but at least 10% of the maximum length of the opening is covered by the longest part of the genital coversheet. In another embodiment, the genital coversheet may extend over the whole length of the opening and may comprise an opening for receiving fecal material.

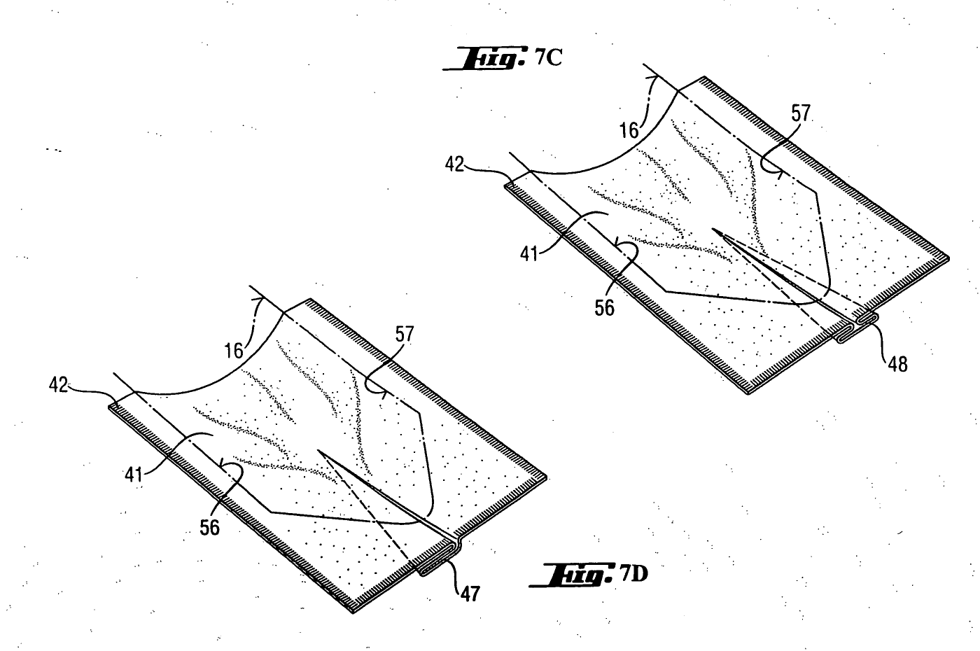

The genital cover sheet may have a substantially transverse edge above, in, or under the opening which is not straight. For example, as shown in FIGS. 1-3, the genital coversheet 41 may have a transverse edge 43 above or under the opening 14, which is not a straight edge, but instead curved or arrow-shaped, to make the genital coversheet 41 more comfortable in use. As shown in FIG. 3, the genital coversheet 41 may have a rounded or V-shaped “transverse” edge 43, i.e., the edge 43 being in the diaper 10 above, in or under the slit opening 14. The transverse edge may be curved or V-shaped (arrow shaped), as shown in FIGS. 1-3, having the center point of the curved edge or of the V-shaped edge closer to the front of the article than the remaining part of the curved edge or V-shaped edge. In some embodiments, the length of the part of the genital coversheet from this center point to the front of the opening may be 10% to 30%, 15% to 25%, or, alternately, 20% to 25% of the maximum length of the opening. The genital coversheet may also have a transverse edge 43 above or under the opening that is substantially straight and perpendicular to the longitudinal axis of the article, as can be seen in FIGS. 7 A-D.

The genital coversheet may be in the form of a pocket. As shown in FIG. 1 for example, the genital coversheet 41 has a partial longitudinal central fold 44 to ensure the genital coversheet 41 can form a pocket in use around the genitals of the user, to protect the genitals from soiling with feces. In certain embodiments, the genital coversheet has one or more substantially longitudinal folds. The folds shorten the width of the genital coversheet material thereby allowing it to shape into a pocket shape. Optionally, (small) parts of the material on one side of the fold or folds may be attached to the part of the (non-folded) genital coversheet, by any know method, to form a fixed fold, i.e., a fold with a bonding area, for example, by use of adhesive, heat or pressure bonding, or, for example, ultrasonic bonding, or a combination of such bonding techniques. Such a fixed fold(s) may have visible bonding areas, like small bonding points.

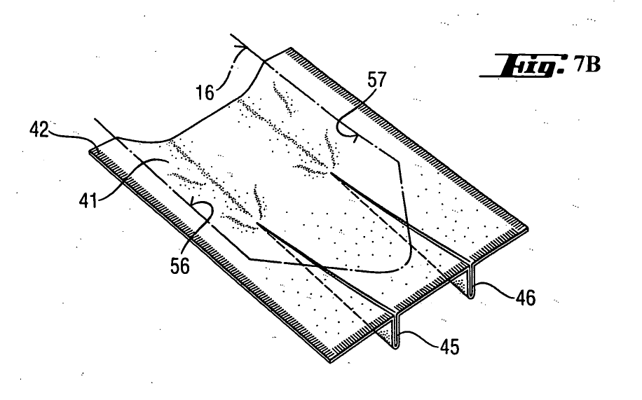

The genital coversheet with the fold or folds may have a substantially square or rectangular circumference in the article or a square or rectangular circumference with a cut-out v-shaped section, as shown in the FIG. 3 and discussed herein in more detail. FIG. 6 shows a perspective view of the shape of a genital coversheet 41 of absorbent article 10, before implementation in the absorbent article 10. FIG. 7A shows a perspective view of the genital coversheet 41 of FIG. 6, which is folded for implementation into the diaper 10 of FIGS. 1 to 3, whereby the areas are shown which in the diaper 10 will become the attachment areas 42; the attachment areas 42 may, for example, comprise adhesive for attachment to the topsheet 11. It is shown in FIG. 7A how, in one embodiment, the genital coversheet 41 will be placed optionally along part of the longitudinal side edges 16 of the opening 14, but in any event along the edges of the cut out section 55, including edges 57 and curved edge 56. The fold 44 in the genital coversheet 41 will ensure that when the genital coversheet 41 is attached in a rectangular or square form with substantially parallel attachments areas 42 to the diaper (e.g., the topsheet 11), the pocket-shape is formed. Thus, by ensuring that the opposing longitudinal side edges are aligned and parallel when attaching the genital coversheet material to the article, and providing the fold or folds herein, the desired pocket shape is formed to protect the genitals from soiling. The folded material 44 may be fixed to part of the remaining genital coversheet material 41 by bonding it thereto, for example, with ultrasonic bonding or welding, to form a fixed fold 44. In some embodiments, the portion of the fold that is bonded/fixed as described herein may have a width of about 0.1 mm to 10 mm, or, alternately, about 0.3 to 5 mm (the width being in the CD direction, perpendicular to the longitudinal axis of the article).

FIGS. 7B, 7C and 7D show alternative folds (44, 45, and 46) that may be used to create a pocket shape in the genital coversheet 41. Obviously, they may vary in width, shape and length depending on the desired pocket shape. The single z-fold 44 as shown in FIG. 7C may be beneficial, because it is easy to process and results in an about flat surface of the genital coversheet, which makes this fold 44 more comfortable in use. The fold 44 may be (partially) bonded to a part of the remaining genital coversheet 41 to form a fixed fold 44 with a bonding area 42, for example, by ultrasonic bonding, and the bonding area 42 may in use comprise visible bonding points, e.g., small dots extending in longitudinal direction along the fold (not shown). The fold or folds also may, for example, be a z-fold, a double or multiple z-folds, an omega-shaped fold, or double or multiple omega-shaped folds, or a w-shaped or multiple w-shaped folds fold. The fold or folds may also be made in the genital coversheet once it has been incorporated into the absorbent article, or prior to incorporation of the genital coversheet material into the absorbent article.

In some embodiments, the genital coversheet material is a three-dimensional activatable material that can be activated and formed into a three-dimensional pocket shape with an activation tool (die) prior to or after incorporation into the article. In other embodiments, the genital coversheet material may be formed into a pocket-shape by guiding it over a guiding tool (rail) while incorporating it into the article. In yet other embodiments, the genital coversheet material may be formed into a pocket-shape by guiding it over a vacuum drum or belt with shapes thereby pulling the genital coversheet material down into shapes and then incorporating the resulting genital coversheet material into the absorbent article. The genital coversheet may also be elastically extendable. In some embodiments, the genital coversheet portion of the topsheet may be extendable in at least the transverse direction. However, the genital cover portion of the topsheet may also be extendable in the longitudinal direction.

The genital coversheets may also be hydrophilic or treated with a hydrophilising agent to render them hydrophilic. Suitable materials for the genital coversheet are nonwoven and woven materials comprising hydrophilic fibers and/ or nonwoven or woven materials treated with a hydrophilising agent, e.g., a surfactant. The genital coversheet 41 may also be urine-permeable, as described herein and it may comprise or consist of a material with micro pores, such as the apertured webs or films described herein, or, for example, the carded webs described herein.

As previously mentioned, the genital coversheet of the absorbent articles may be urine permeable. Urine permeability can be determined by the Strike Through test, Edana test method 150.3-96, which is modified in that it applies 2 gushes of 5 ml at the same sample with a waiting time of 60 seconds between 2 gushes (without changing the absorbent pad) and in that the absorbent pad consists of 10 plies of filter paper, rather than 5 plies; the filter paper used may be Ahlstroem grade 989, or equivalent; the pick-up paper may be Ahlstroem Grade 632 or equivalent. This modified Edana test method provides the strike through times of the first gush and second gush, which are each an average of the values obtained in 3 tests.

In some embodiments, the genital coversheet may have an average urine permeability of the first gush strike through time of less than 7 seconds, less than 5 seconds, or less than 3 seconds. The first gush strike through time may be between 1 and 3 seconds or between 1 and 2 seconds. The genital coversheet may have a second gush strike through time of less than 9 seconds, less than 7 seconds, or less than 5 seconds. The second gush strike through time may be between 1 and 7 seconds, between 2 and 5 seconds, or between 1 and 5.

In certain embodiments, the genital coversheet does not allow moisture to pass back to the skin, and thereto, it may have a low rewet. This can be determined by the Wetback Test, Edana test method 151.3-02, under 23° C. and 50% humidity test conditions, as specified in the Edana test, and using a filter paper with a loading factor of 3.30, as specified therein, and resulting in average rewet values, which are an average of 3 test results (the filter paper used may be as specified above). The genital coversheet may have an average rewet value of less than 0.8 g, less than 0.5 g, less than 0.3 g, less than 0.2 g, or less than 0.15 g.

In certain embodiments, the genital coversheet may allow low viscosity fecal material pass through and entangle it, thereby protecting the genitals from the fecal material. The genital coversheet may be formed from materials with large pores and large caliper (e.g., having a mean pore size of 50 to 400 microns or from 100 to 300 microns and an average caliper (measured under compression of 0.3 psi) of 0.5 mm to 1.5 mm or 0.7 mm to 1 mm such as for carded resin-bonded or air-through nonwovens made of, for example, PET fiber).

The genital coversheet may be soft to touch. In some embodiments, at least the surface in contact with the genitals is formed by a carded or spunbond material. Materials for the genital coversheet may include spunbond nonwovens, laminates of spunbond nonwovens (S) and meltblown nonwovens (M), such as SM and SMMS laminates, carded nonwovens, or apertured formed films, such as, for example, described in WO96/00549, which each may be coated with a hydrophilising agent, e.g., a surfactant.

In embodiments using carded nonwoven materials, the material may have an average calliper (measured under compression of 0.3 psi) of at least 0.3 mm, at least 0.5 mm, or at least 0.7 mm, and/or a mean pore size of from 100 to 300 microns.

The genital coversheet or a part thereof may also be colored (e.g., other then the normal white color of known diapers) with a coloring agent and/or may be made opaque by having an opacifying agent. Color and opacity help to mask the fecal material deposited in the absorbent article and present under the genital coversheet.

A topsheet with an opening, typically along its longitudinal axis, and primary elasticated areas and a genital coversheet may be obtained according to the following method.

A longitudinally extending (slit) opening may be formed (cut) in the topsheet material first and a pair of primary elastic bands are attached, in stretched state, to the topsheet, so that an elastic band is present adjacent or in close proximity of each longitudinal edges of the opening. Alternatively, the primary elastic bands are first attached to the topsheet in stretched state, extending longitudinally over the topsheet, for example, an X-shape, whereafter a longitudinally extending (slit) opening is formed (cut) through part of the topsheet material and part of the elastic bands, typically along the longitudinal axis of the topsheet material and the strands or bands.

Suitable adhesives for attaching the primary elastic strands or bands may include H2031, available from ATO-Findley and HL-1620 available from H.B. Fuller (St Paul, USA).

In a non-limiting example, two elastic band of L-89 elastic material, available from Fulflex, with (in relaxed state) a thickness of about 0.1 mm, a width of 20 mm and a length of 17 cm, are obtained and also a sheet of a polypropylene nonwoven material, which is 20-25 cm wide and 50-55 cm long, such as available from BBA, Fibertex or Pegas.

In stretched state, a slit opening with a length of between 25 cm and 35 cm, typically about 28 cm to 32 cm is cut in the nonwoven. This may be cut as a slit, whereby the slit opening obtains its width due to the spreading elasticated areas, or the slit opening may be cut with a width dimension, for example, up to 4.0 cm. Typically, the slit opening is cut as a slit without width dimension, but in the front region of the topsheet, end the front point of the slit opening, an additional diamond shaped cut is made, as can be seen in FIG. 4.

If the elasticated areas are present on the surface of the topsheet which is in contact with the skin of the user, an additional layer, e.g., nonwoven material, may be placed on the elastic areas, to avoid direct contact by the elastic areas with the skin.

One elastic band is glued in a partially stretched state to each longitudinal edge of the opening of the nonwoven. This is, for example, done such that the center 9 cm of the elastic band is stretched 336%, and the end portions of each 4 cm are maintained in unstretched state. Each elastic band is then glued on the non-woven in a manner that the front end region of one elastic band bends away from the front end region of the opposing elastic band, and the back end region of one elastic band bends away from the back end region of the opposing elastic band, in the shape of an X. This is done such that, after application, the transverse distance between to the inner two edges of the front end regions and of the back end regions is, for example, 80 mm in stretched state. The distance between the center point on the elasticated areas is, for example, only 40 mm.

The angle of the front end region with the line through the center portion and parallel to the longitudinal axis of the topsheet, as described above and can be seen in FIGS. 2 and 3 as angle α, is, for example, about 10° to 40°, as described herein. The angle of the back end region, as described above and can be seen in FIG. 2 as angle β, is also, for example, about 10° to 40°.

Then, the genital coversheet may be attached to the topsheet in stretched state, such that the front 15% to 35% of the maximum length of the opening is covered by the longest part of the genital coversheet. The genital coversheet may have a curved or V-shaped edge above the opening. The genital coversheet may, for example, have the shape shown in FIGS. 1-3, and it may be folded prior to attachment to the topsheet, for example, as shown in FIG. 6. However, the transverse edge may be substantially straight and perpendicular to the longitudinal axis of the article, as can be seen in FIGS. 7A-C.

The genital coversheet may, for example, be attached to the surface of the topsheet which is not in contact with the skin of the user, with at least two longitudinal areas (lines, spirals, dots) of adhesive, each being between a longitudinal edge of the topsheet and an elastic band, e.g., about 0.5 to 1.5 cm spaced away from the elastic band, and with a V-shaped attachment area, as shown in FIG. 6. In some embodiments, the distance between the two substantially parallel attachment areas may, for example, be at the most about half the width of the genital coversheet between these areas, so that the genital coversheet hangs downwards in use, under the opening, as a pocket for the genitals.

The topsheet may be used on a Pampers Premium Size 4 diaper, designed for a baby weight range of 21-37 pounds, or it may replace the layer of a Pampers premium Size 4 diaper which is on the absorbent core and in use in contact with the skin of the baby. Thereto, the topsheet with the elastic bands may be attached to the front and back waistbands, and typically to the longitudinal side edges of the backsheet.

The articles (e.g., diaper) when packed in their packaging material, may include two transverse folds, so that when unfolded for use by the user or care taker, the article (e.g., diaper) is in a U-shape and easier to apply.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a backsheet; an absorbent core; a topsheet provided with at least one opening adapted to receive fecal material, the topsheet and the opening thereof each having a front region and a back region, wherein a void space is present between the absorbent core and the topsheet; and a genital coversheet positioned in, under or above the front region of the opening, the genital coversheet having one or more folds or partial folds to form a pocket that in use covers the genitals.

2. The absorbent article of claim 1, wherein the genital coversheet comprises one or more (partial) folds that extend in a substantially longitudinal direction.

3. The absorbent article of claim 2, wherein the folds or partial folds are fixed folds.

4. The absorbent article of claim 1, wherein the genital coversheet is urine permeable and the topsheet is urine-impermeable.

5. The absorbent article of claim 1, wherein the genital coversheet is positioned between the topsheet and the absorbent core, and wherein the cover sheet is attached to the topsheet, but not attached to the absorbent core.

6. The absorbent article of claim 1, wherein the genital coversheet comprises a single central longitudinal fixed fold or a single central longitudinal fixed partial fold.

7. The absorbent article of claim 1 wherein the genital coversheet is formed from a sheet with substantially parallel longitudinal side edges, which are attached to the topsheet with substantially longitudinally extending parallel opposing attachment areas.

8. The absorbent article of claim 1, wherein the opening has two longitudinal side edges and parts of the genital coversheet are attached to the topsheet along the longitudinal side edges of the opening and one or more parts of the genital coversheet are not attached to the absorbent article and form a pocket between the opening.

9. The absorbent article of claim 8, wherein the opening in the topsheet is a slit opening that has two longitudinal side edges and the topsheet comprises elasticated areas along at least part of the longitudinal side edges of the opening.

10. The absorbent article of claim 8, wherein the topsheet comprises elasticated areas and non-elasticated areas, wherein the genital coversheet is attached to the topsheet in the non-elasticated areas of the front region of the topsheet.

11. The absorbent article of claim 1, wherein the genital coversheet comprises a spunbond nonwoven web, carded nonwoven web, or a formed film.

12. The absorbent article of claim 1, wherein the maximum length of the part of the genital coversheet that is present above, in or under the opening is from 10% to 50% of the maximum length of the opening.

13. The absorbent article of claim 1, wherein the genital coversheet comprises a carded nonwoven with an average calliper (measured under compression of 0.3 psi) of at least 0.3 mm and with a mean pore size of from 100 to 300 microns.

14. The absorbent article of claim 1, wherein the absorbent article is an adult or infant diaper or training pants.

15. A process for providing an absorbent article comprising a backsheet, an absorbent core, and a topsheet with at least one opening adapted to receive fecal material, a void space between the absorbent core and the topsheet, and a genital coversheet in the form of a pocket, the genital coversheet being positioned in, under or above part of the opening, the process comprising the step of: shaping a genital coversheet (material) into a pocket shape by vacuum forming, shaping it with a shaping tool, or by providing one or more folds or partial folds or fixed folds.

16. A process for making an absorbent article as in claim 1, wherein a genital coversheet material is provided with one or more folds or partial folds, which may optionally be attached to part of the non-folded genital coversheet material to form a fixed fold or folds, prior or subsequently to attaching the genital coversheet material to the topsheet.

17. The process of claim 16, wherein the attachment is done by attaching substantially longitudinally parallel extending side edges of the genital coversheet material to the topsheet with substantially longitudinally extending, parallel opposing attachment areas.
